(19) United States
(12) Patent Application Publication
(10) Pub. No.: US 2022/0054327 A1
(43) Pub. Date: Feb. 24, 2022

(54) Device For Male Urinary Incontinence

(71) Applicant: HEALTH ITALIA S.R.L., Latina (IT)

(72) Inventor: Cristian COLUZZI, Latina (IT)

(21) Appl. No.: 17/418,411

(22) PCT Filed: Dec. 27, 2019
(86) PCT No.: PCT/IT2019/050280

§ 371 Date: Jun. 25, 2021

(30) Foreign Application Priority Data
Dec. 28, 2018 (IT) 102018000021289

(51) Int. Cl. A61F 13/491; A61F 13/496; A61F 13/505; A61F 13/475

(52) U.S. Cl. CPC A61F 13/4915; A61F 13/496; A61F 2013/51047; A61F 13/475; A61F 13/505

(57) ABSTRACT

Device for male urinary incontinence, comprising at least one pair of underpants which at least one through hole (18) is made, into which the urogenital apparatus (19) is introduced, and at least one absorbent aid (12) provided with at least one bag (21) and connected to the external surface (13) of the underpants (11) by means of support and attachment means (14, 17, 17′); the absorbent aid (12) is provided with at least one through hole (20) substantially aligned with the through hole (18) of the underpants (11) to allow the introduction of the urogenital apparatus (19) inside said bag (21) of the absorbent aid (12).

### FIELD OF THE INVENTION

The present invention concerns a device for male urinary incontinence.

### BACKGROUND OF THE INVENTION

Urinary incontinence is normally understood as the inability to hold urine due to anatomical or functional alterations of the sphincters of the bladder. Incontinence is a real geriatric syndrome and the the risk factors typically include age-related physiological changes, comorbidities, medications taken and above all functional alterations.

Urinary incontinence can generally be categorized as:
stress or effort incontinence, which occurs when an even minimal effort is made, such as a sneeze, laughter or other;
urge incontinence, which is associated with an urgent urge to urinate so that if it is not possible to reach the bathroom in time, the bladder can empty at any moment;
mixed incontinence, which occurs when stress and urge incontinence coexist;
reflex incontinence, which manifests with more or less abundant losses followed by a strong and constant desire to urinate again;
neurogenic incontinence, which occurs when the coordination between the brain, spinal cord and bladder fails;
overflow incontinence, which occurs when there is a sphincter injury;
enuresis, which occurs when urine is lost during the night.

To date, according to the World Health Organization, there are at least 200 million people in the world suffering from urinary incontinence. Incontinence is a condition that affects all age groups; however, it is difficult to gather precise data on its prevalence since many people who are afflicted with it do not raise the problem with their doctor, especially because of embarrassment. It is estimated that in the population aged between 15 and 64 years the prevalence in men varies between 1.5 and 5%, and in women between 10 and 30%.

Male urinary incontinence can be classified substantially into three categories, namely:
alteration of the sphincter muscle mechanism; this category comprises incontinences following surgery/endoscopy (mostly due to prostate cancer but also after reconstruction of the bladder, interventions for benign prostate diseases and radiotherapy), after traumas of the pelvis and incontinence that manifests itself in pediatric age;
alteration of the bladder function; this category comprises incontinences linked to incompatible hyperactivity of the bladder and/or structural alterations;
losses through non-natural pathways; this category comprises incontinences involuntarily linked to the presence of anatomical anomalies that arise after particular pathologies or operations on the lower urinary system (fistulae).

Male urinary incontinence is linked to various factors that sometimes combine, so that, for adequate therapy, it is necessary to determine a precise diagnosis based on clinical and instrumental data. The foundations of the diagnosis refer in the first place to an accurate overview of the patient's clinical history, which has to take into account not only references to the urinary system, but also to current diseases and treatments that can interfere with the function of the bladder and of the urethra.

It is known that various diseases (neuropathies, diabetes, operations on the lower abdomen) can, to varying degrees, alter the function of the urinary system, causing continuous or temporary incontinences of varying intensity and which can sometimes be controlled with specific therapies. A second fundamental moment in the diagnosis of male incontinence is clinical examination. During the examination, the specialist has to highlight if there are changes in the urinary system and/or in the nerve pathways that regulate the bladder and the ability to control the urine (pelvic floor muscles). The clinical examination is called a neuro urological test.

The specialist also assesses whether the patient is able to empty the bladder completely or only partly, due to urine remaining after urination (bladder retention).

Urine has to be examined in order to check if there are infections in progress or signs attributable to the presence of pathologies of the urinary system that can alter its function (for example, stones or tumors).

The collaboration of the patient is of fundamental importance, since he must not only precisely report the symptoms, but also accurately record the progress of the bladder function throughout the day, marking on specific questionnaires the individual urinations, during the day and night, the losses of urine, possibly quantified, and the sensations experienced. It is also useful to record the amount of liquid taken during the day and the ongoing drug therapies that can interfere with the function of the bladder and of the urethra (urination diary).

The subsequent evaluation provides an objective demonstration of incontinence and an evaluation of its extent following pre-established tests in a clinic (pad test), in order to confirm and better specify what the patient has already demonstrated with the urination diary previously completed, for example at home or in the workplace, that is, during his normal everyday life. Following this, instrumental tests can be provided which tend to highlight possible alterations of the urethral tube and bladder with direct observation (urethrocystoscopy) in order to rule out the presence of narrowing of the urethra (stenosis), bladder pathologies (stones, tumors), enlargement of the prostate and alterations of the bladder wall which can justify functional behavioral anomalies.

The main examination in evaluating male incontinence is the urodynamic investigation which, by measuring the pressures in the bladder and urethra during the steps of filling and emptying the bladder, is able to reveal what is, or are, the cause, or causes, of the incontinence reported by the patient.

The isolated test, or associated with a radiological evaluation (video urodynamic test), has to be conducted with precision and evaluated by specialists able to provide an overall judgment on the patient's situation, taking into account what emerges from the urodynamic test and from the analysis of previous assessments (symptoms, urinary diary, clinical examination).

The diagnosis of male incontinence is a fine diagnosis, which requires experience from the operator, but able to direct therapy precisely.

There are several remedies that try to counter male urinary incontinence, but often these remedies are not very effective and appropriate.

Products for urinary incontinence can be used as the only treatment in case of failure of other forms of therapy, pharmacological or surgical, of the disorder or as additional support for other interventions of the pharmacological type.

The known products used to manage incontinence can be divided into: disposable absorbent products; external catheters (or condoms); and collection bags.

It should be remembered that the use of these products cannot, and must not, lead to disregarding other interventions to promote the voluntary control of the sphincter. According to the data provided by the most recent literature, the use of adult absorbent diapers causes a certain dependence and in about 70% of cases causes a reduction in the motivation to control the sphincter with other techniques.

It is therefore important, on the one hand, to help the subject to reach the bathroom in good time (accompanying the subject to the toilet at regular time intervals, or facilitating the identification of the bathroom door for subjects who are confused or have cognitive disorders), on the other hand, it can be useful to regularly make the subject practice exercises for the pelvic floor (especially in case of stress incontinence) which consist in contracting the pelvic muscles several times a day (for example 8 contractions, 3 times a day).

To date there are no comparative studies of the effectiveness of one adult absorbent diaper compared to another; however, when it is necessary to choose the absorbent product, it is necessary to consider both the type of incontinence, the severity, the motor skills of the subject and more generally the lifestyle of the subject.

Absorbent products comprise both incontinence pads, which are worn instead of normal underwear, and also sanitary towels to be worn under normal underwear. All absorbent products contain inside them polymers that are able to turn into gel when in contact with urine.

The surface remains dry, preventing contact between the skin and the gel and reducing the risk of skin maceration. Absorbency varies from product to product and ranges from 60 to 2000 ml. The main types of absorbent products available are:

rectangular pads, which have poor absorbent capacity therefore can be useful in case of mild urinary incontinence but are not recommended for men since their shape is not suitable for the male anatomy;

pads for light incontinence, which are worn with normal underwear;

shaped pads for medium to severe incontinence, which are of different sizes and can be used with one's own underwear or with a pair of elastic attachment underpants which guarantee greater stability;

elasticized absorbent panties, which are worn as normal underwear;

shaped adult diapers for medium to severe incontinence, which come in different sizes and a person can use with his/her own underwear or with a pair of elastic attachment underpants which guarantee greater stability;

elasticized absorbent underpants, which are worn as normal underwear;

underpant-shaped adult diapers which close with lateral adhesives, or with a belt, or with Velcro, which are generally reserved for bedridden subjects.

The adult diaper should be changed as soon as it is known to be dirty, in any case it has to be replaced every 3 hours, except at night when it can be kept on for longer in order to not interrupt the subject's sleep. Frequent changes help reduce the risk of skin irritations.

It is important to regularly inspect the skin in the perineal area in all incontinent subjects, in order to detect signs of dermatitis resulting from incontinence. In particular, it is necessary to verify that there are no erythematous-desquamative or crusty lesions, or vesicles.

The skin should be cleaned after each urination in order to avoid prolonged contact with the urine. Topical agents that act as a barrier can be used in order to keep the skin dry.

Any lesions that have already formed have to be protected from urinary contamination with water-repellent dressings.

Condoms (or catheters) are sheaths made of latex or synthetic material equipped with a discharge tube and connected with a collection bag, and are a valid alternative to internal catheterization. They are available in different sizes (the more precise the measurement, the greater the adhesion) and they are easily applicable by the patient himself In order to promote the adhesion of the male urogenital apparatus, they can be attached with mastic or adhesive strips. The skin has to be prepared for the attachment with scrupulous personal hygiene. The use of oily or creamy emollients that reduce the adhesiveness of the adhesives used must be avoided, as well as overly aggressive soaps that can irritate and excessively dry the skin. The condom has to be replaced daily and the patient has to be taught how to check the urogenital apparatus: cases of maceration or irritation are in fact frequent, caused by the rubbing of the glans against the internal end of the condom. Cases of urinary tract infection are not uncommon, although the incidence is lower than the infections found with the use of the transurethral catheter.

The collection bags allow to collect urine over the course of 24 hours. Substantially, two types exists on the market: those for use in bed, indicated for patients who do not move and characterized by a longer connection tube; and leg bags, smaller and with a shorter tube, which are clamped to the patient's leg or thigh. Most of these bags have a non-return valve mechanism inside them that prevents the urine from rising back into the bladder, thus reducing the risk of bacterial contamination. Only devices with a non-return valve can be re-used.

As we have seen, there are several products available on the market aimed at combating male urinary incontinence, however, often these known products present various problems of hygiene and of use, as well as often proving unsuitable for users' needs, which can be linked, for example, to a particular disturbance but also to normal daily activities.

For example, document U.S. Pat. No. 8 702 667 B1 describes a device for male urinary incontinence comprising an undergarment provided with a front flap stably attached to the undergarment with respect to a lower side in order to selectively allow access to a front pocket where a pad is positioned. Both the undergarment and also the pad have a corresponding hole for the passage, during use, of the male urogenital apparatus. In particular, the front flap is stably attached to the undergarment with respect to its lower side only, and allows it to act as a pocket for containing the pad only after it has been attached to the undergarment by means of suitable attachment devices.

One of the disadvantages of the solution described in document U.S. Pat. No. 8 702 667 B1 is that changing the pad is complicated because it is necessary to open the front flap with the consequent risk of getting dirty during the operation. In addition, the opening of the front flap requires lowering the pants and the front flap, once opened, is no longer able to support the pad which, in the event of excessive weight, could detach from the undergarment.

Another disadvantage of the solution described above is that the pad is totally made of absorbent material, therefore it does not seem to have gripping points, for example for its removal, that are definitely clean. Consequently, replacing the pad is not very hygienic.

Moreover, the attachment means of the pad for attaching it to the undergarment are disposed around the hole of the pad itself, this being able to cause contact, and therefore possible irritation, with the patient's pubic area or with the urogenital apparatus which passes inside it.

Furthermore, the absorbent device described in U.S. Pat. No. 8 702 667 B1 does not appear to have an elasticized hole, therefore undesired urinary refluxes on the undergarment are possible.

Document US 2003/0028161A1 describes a containment bag, not an absorbency one, attached outside an undergarment by means of suitable attachment means. However, this solution is not very stable, with the high risk that the bag may detach from the undergarment. Furthermore, this device is very uncomfortable for the patient, highly visible and unsightly.

WO 2013/004131 A1 describes a condom made of semi-rigid plastic material provided with closing and emptying means. This device has a minimal containment capacity and the contact of the male urogenital apparatus with the urine contained in it can cause bacterial proliferation and, in the more severe cases, even maceration of the skin. Furthermore, the removal, replacement or washing of the condom is not very hygienic.

Other limitations and disadvantages of conventional solutions and technologies will be clear to a person of skill after reading the remaining part of the present description with reference to the drawings and the description of the embodiments that follow, although it is clear that the description of the state of the art connected to the present description must not be considered an admission that what is described here is already known from the state of the prior art.

There is therefore the need to perfect a device for male urinary incontinence that can overcome at least one of the disadvantages of the state of the art.

One purpose of the present invention is therefore to provide a device for male urinary incontinence devised specifically to meet the various and multiple needs of incontinence, which takes into consideration the social life and normal daily activities of the user, which is substantially invisible, which does not limit movement, which is hygienic and antibacterial, which is practical both during use and also when being changed, which respects the morphology of the male urogenital apparatus, which protects the areas of the urogenital apparatus from possible rashes caused by friction or even worse from situations in which the skin remains damp and wet, with consequent development of dermatitis or skin lesions, and which allows to avoid using bags, catheters or suchlike.

The Applicant has devised, tested and embodied the present invention to overcome the shortcomings of the state of the art and to obtain these and other purposes and advantages.

### SUMMARY OF THE INVENTION

The present invention is set forth and characterized in the independent claim. The dependent claims describe other characteristics of the invention or variants to the main inventive idea.

In accordance with the above purposes, the invention concerns a device for male urinary incontinence, comprising at least one pair of underpants in which at least one through hole is made, into which the urogenital apparatus is introduced, and at least one absorbent aid provided with at least one bag and connected to the external surface of the underpants by means of support and attachment means. The absorbent aid is provided with at least one through hole substantially aligned, during use, with the through hole of the underpants to allow the introduction of the urogenital apparatus inside the bag of the absorbent aid.

The underpants comprise, on the external surface, at least one pocket having an upper entry side for the insertion of a lower first portion of the absorbent aid. The absorbent aid comprises an upper second portion outside the pocket which is connected to the external surface of the underpants by means of removable attachment means.

Advantageously, through the use of an absorbent aid applied outside a pair of underpants and equipped with a bag where the urogenital apparatus is positioned, the present device proves to be particularly effective and suitable to respond to the different and multiple needs of incontinence. The present device also takes into account the user's social life and normal daily activities, is substantially invisible, does not limit movement, is hygienic and antibacterial, is practical both during use and also when being changed, it respects the morphology of the male urogenital apparatus, it protects the areas of the urogenital apparatus from possible rashes due to friction, or even worse from situations in which the skin remains damp and wet and allows to avoid using bags, catheters or suchlike.

In some embodiments, at least one of either the through hole of the underpants or the through hole of the absorbent aid can be provided with a membrane on which a plurality of cuts are defined, passing through the center of the through hole and defining a coherent plurality of membrane sectors configured to allow the selective introduction of the urogenital apparatus inside the bag of the absorbent aid.

This solution has the advantage that, in inactive conditions, the through hole is substantially closed, while when the urogenital apparatus exerts a thrust on it, it opens in an elastic manner in order to perform its function.

The through hole of the underpants and/or the through hole of the absorbent aid can comprise an elasticized edge, in order to increase stability and seal against unwanted refluxes. This elasticized edge can, for example, be defined by an elastic stitching.

According to another aspect of the invention, the absorbent aid can comprise, from inside to outside, at least one layer of antiallergic and antibacterial material and at least one layer of pressed cellulose with high carrying capacity.

According to other aspects of the invention, the absorbent aid can comprise, from inside to outside, at least one layer of antiallergic and antibacterial material, at least one layer of pressed cellulose with high carrying capacity and at least one layer of pressed cellulose made of super-absorbent polymers.

In other embodiments, the absorbent aid can comprise, from inside to outside, at least one layer of antiallergic and antibacterial material, at least one layer of pressed cellulose with high carrying capacity, at least one layer of pressed cellulose made of super-absorbent polymers and at least one layer of hypoallergic-antibacterial pressed cellulose.

In the present device, at least the absorbent aid can be externally coated by at least one waterproof external layer, for example made of non-woven polypropylene fabric, water-repellent non-woven fabric or suchlike.

According to another aspect of the invention, the absorbent aid can comprise at least one pair of wings on which the removable attachment means are positioned. The wings as above are attached on one side and on the other of the second portion, since they are disposed outside the bulk of the second portion.

According to another aspect of the present invention, the pocket can be provided, on the entry side, with an elasticized edge in order to increase the effectiveness of the positioning of the absorbent aid.

The invention also concerns a pair of underpants, comprising a through hole into which the urogenital apparatus is introduced and usable in a device for male urinary incontinence.

The invention also concerns an absorbent aid, provided with at least one bag, at least one through hole to allow the introduction of the urogenital apparatus inside the bag and usable in a device for male urinary incontinence.

These and other aspects, characteristics and advantages of the present disclosure will be better understood with reference to the following description, drawings and attached claims. The drawings, which are integrated and form part of the present description, show some embodiments of the present invention, and together with the description, are intended to describe the principles of the disclosure.

The various aspects and characteristics described in the present description can be applied individually where possible. These individual aspects, for example aspects and characteristics present in the description or in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example with reference to the attached drawings wherein:

FIG. 1 is a front view of a device for male urinary incontinence according to the present invention;

FIG. 2 is a schematic longitudinal section view of the present device for male urinary incontinence;

FIGS. 2a, 2b, 2c are three section views and on a larger scale of different types of absorbent aid of the present device for male urinary incontinence;

FIG. 3 is a front view of a pair of underpants of the present device for male urinary incontinence;

FIG. 4 is a front view of an absorbent aid of the present device for male urinary incontinence;

FIG. 5 is a rear view of the absorbent aid of FIG. 4;

FIG. 6 is a variant of FIG. 4;

FIG. 7 is an enlarged detail which shows a variant of the through hole of the absorbent aid and/or of the through hole of the underpants.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We will now refer in detail to the various embodiments of the present invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

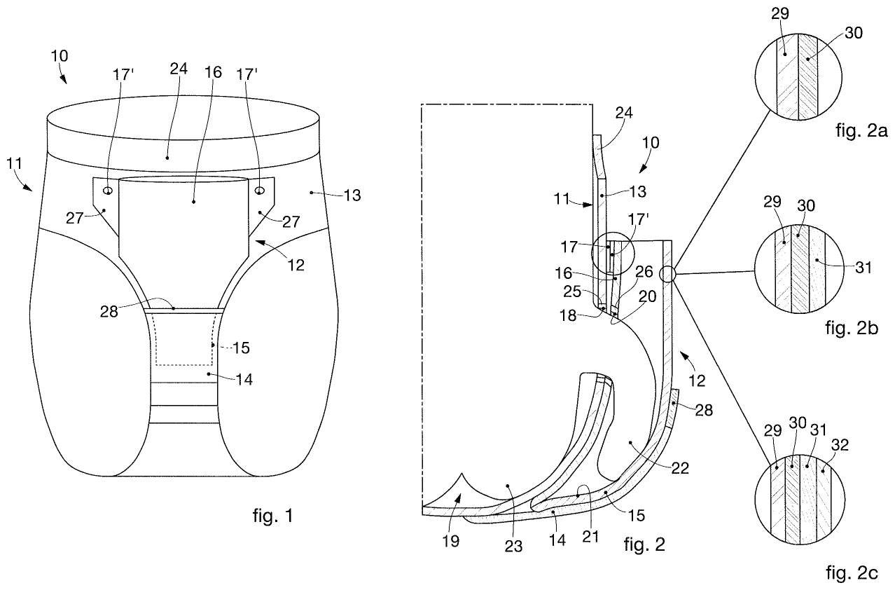

With reference to the attached drawings and in particular to FIG. 1 thereof, a device 10 for male urinary incontinence according to the present invention comprises a pair of underpants 11 to which an absorbent aid 12 is applied, such as a pad or suchlike.

The underpants 11 are preferably made of antiallergic elasticized cotton and are provided on the external surface 13 with a pocket 14 having an upper entry side for inserting a first lower portion 15 of the absorbent aid 12. The pocket 14, therefore, is stably attached to the underpants 11 on three sides, a lower side, opposite the upper side as above, and with respect to two sides disposed along the flanks of the pocket 14, leaving the upper side open and therefore free for the insertion of the first portion 15 of the absorbent aid 12. In this way, the pocket 14 is always defined for the support and correct positioning of the absorbent aid 12.

The absorbent aid 12, during use, is disposed resting with respect to the first portion 15 inside the pocket 14 which, therefore, allows to at least partly support the absorbent aid 12.

The pocket 14 can be made of elasticized cotton and can also be provided, on the upper open entry side, with an elastic 28. The elastic 28 allows the absorbent aid 12 to be positioned correctly and in a substantially stable manner inside the pocket 14.

The absorbent aid 12 comprises a second upper portion 16 which remains outside the pocket 14 and which is connected to the external surface 13 by means of removable attachment means 17, 17′ which can be snap-in or pressure buttons provided both in the absorbent aid 12 and also in the underpants 11, attachment systems with Velcro, also provided both in the absorbent aid 12 and also in the underpants 11, or other.

The removable attachment means 17, 17′ and the pocket 14 therefore represent one of the possible modes for the underpants 11 to attach to, and to support the absorbent aid 12, therefore they are configured as support and attachment means and suitable to connect the absorbent aid 12 to the external surface 13 of the underpants 11. In particular, the pocket 14, acting as a support for the absorbent aid 12, allows to unload part of the weight of the absorbent aid 12 from the removable attachment means 17, 17′ preventing these from dragging the edge of the underpants 11 downward. Furthermore, the attachment means 17, 17′ are disposed far from the through hole 20 and therefore far from the patient's pubic area in order to avoid any possible contact with it.

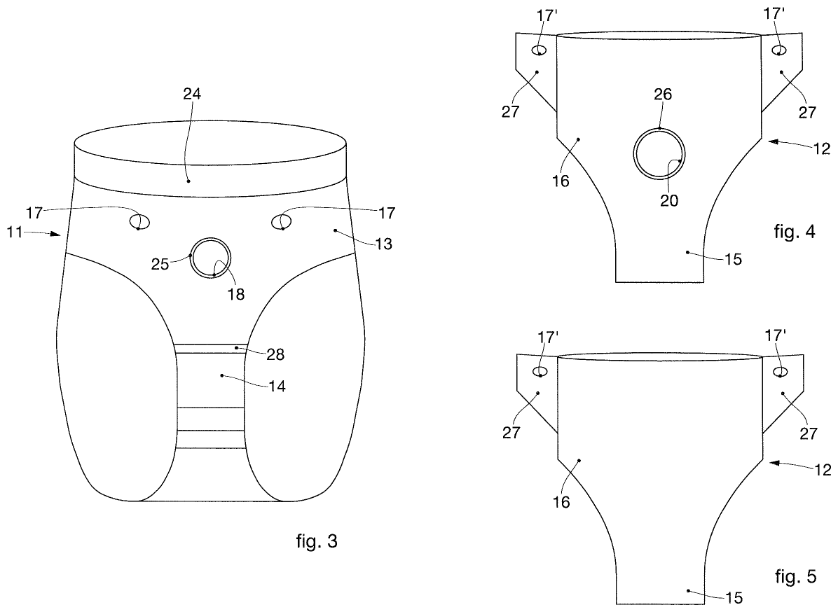

The underpants 11, see FIG. 2 or FIG. 3, comprise a through hole 18 from which the urogenital apparatus 19, in particular the penis 22, can exit. This through hole 18 can comprise an elasticized edge 25 which allows high stability and prevents the reflux of urine.

The absorbent aid 12 is also provided with a through hole 20 that allows to position the urogenital apparatus 19 at least partly inside a bag 21 of the absorbent aid 12.

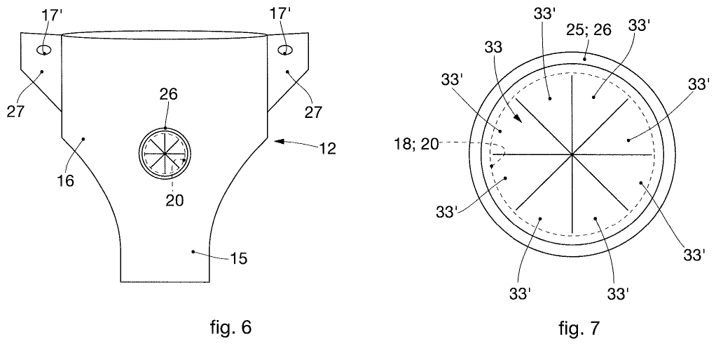

In accordance with a possible embodiment, shown in FIGS. 6-7, the through hole 20 of the absorbent aid and/or the through hole 18 of the underpants 11 can have a circular shape and be provided with a membrane 33 on which a plurality of cuts are defined, passing through the center of the through hole 20 and/or of the through hole 18. The through cuts define a coherent plurality of membrane sectors 33′ configured to allow the selective introduction of the urogenital apparatus 19, in particular of the penis 22, inside the bag 21 of the absorbent aid 12. This solution can guarantee a further barrier against possible refluxes, effectively increasing the safety and hygiene of the device 10.

The bag 21 is formed at least in the first portion 15 of the absorbent aid 12, see for example FIG. 2 and FIG. 4. The through hole 20 can comprise an elasticized edge 26 which allows high stability and prevents the reflux of the urine.

In accordance with possible embodiments, at least one of either the elasticized edge 25 of the underpants 11 or the elasticized edge 26 of the absorbent aid 12 can be defined by an elastic stitching. The elastic stitching allows to increase the degree of wearability and comfort of the device 10.

The through hole 20 of the absorbent aid 12 is substantially aligned with the through hole 18 of the underpants 11, so as to allow the passage of the urogenital apparatus 19, in particular of the penis 22, inside the bag 21.

As can be seen from FIG. 2, by means of the pocket 14 and the bag 21, the penis 22 of the urogenital apparatus 19 can be kept separate from the testicles 23, in order to prevent rubbing and reciprocal direct contacts, which could lead to phenomena of irritation or even skin maceration. The bag 21 also allows to prevent urinary reflux phenomena.

The underpants 11, see also FIG. 3, comprise in the upper part an elastic 24 of suitable size, which allows a comfortable and stable wearability thereof.

The absorbent aid 12, see also FIG. 4 and FIG. 5, comprises at least one pair of wings 27 on which the removable attachment means 17′ are positioned. The wings 27 are attached on one side and on the other of the second portion 16, being disposed outside the overall bulk of the second portion 16. The presence of the wings 27 and their disposition allows to define a pair of gripping points that are definitely dry, and therefore not impregnated with urine, which allow a hygienic removal of the absorbent aid 12 so that it can be replaced.

The absorbent aid 12 can be externally coated by at least one waterproof external layer, for example made of non-woven polypropylene fabric, water-repellent non-woven fabric or similar. This external layer can be equipped with elastic threads, and on the sides it can be equipped with hypoallergenic and water-repellent elastic barriers against the leakages of urine.

The absorbent aid 12, and in particular the zone of the bag 21, as can be seen in the enlarged sections of FIGS. 2a, 2b and 2c, can be made by means of a multi-layered structure: in the case of mild incontinence pathology, the absorbent aid 12 can provide, from the inside to the outside, at least one layer 29 of antiallergic and antibacterial material and at least one layer 30 of pressed cellulose with high carrying capacity, FIG. 2a.

In the case of medium incontinence pathology, the absorbent aid 12 can provide, from the inside to the outside, at least one layer 29 of antiallergic and antibacterial material, at least one layer 30 of pressed cellulose with high carrying capacity and at least one layer 31 of pressed cellulose made of super-absorbent polymers, FIG. 2b.

In the event of severe incontinence pathology, the absorbent aid 12 can provide, from the inside to the outside, at least one layer 29 of antiallergic and antibacterial material, at least one layer 30 of pressed cellulose with high carrying capacity, at least one layer 31 of pressed cellulose made of super-absorbent polymers and at least one layer 32 of hypoallergic-antibacterial pressed cellulose, FIG. 2c.

The absorbent aid 12 can also provide at least one waterproof barrier layer made of water-repellent non-woven fabric.

Advantageously, as described, the present device 10 for male urinary incontinence is specifically designed to meet the different and multiple needs of incontinence, it takes into account the social life and normal daily activities of the user, it is substantially invisible, it does not limit movement, it is hygienic and antibacterial, it is practical both during use and also when being changed, it respects the morphology of the male urogenital apparatus, it protects the areas of the urogenital apparatus from possible rashes due to friction, or even worse, from situations in which the skin remains damp and wet, with consequent development of dermatitis or skin lesions, and it allows to avoid using bags, catheters or suchlike.

In addition, the present device 10 for male urinary incontinence allows, where there is a minimal incontinence or a stress incontinence, to practice sport in complete safety and discretion.

Where there is a minimal incontinence or a stress incontinence, this device 10 in fact can advantageously be changed quickly and hygienically.

The present device 10 for male urinary incontinence also proves to be very comfortable, invisible and stable even in the event daily activity involves long periods of driving a car, or in any case sitting to perform one's job.

By means of the present device 10 for male urinary incontinence, therefore, the user can feel comfortable at any time both during the day and also during the night, without the recurring problem of the product moving or the urine leaking.

Changing the present device 10, moreover, is simple and fast both in an upright situation and also in a decubitus situation.

It is clear that modifications and/or additions of parts may be made to the device for male urinary incontinence as described heretofore, without departing from the field and scope of the present invention.

It is also clear that, although the present invention has been described with reference to some specific examples, a person of skill in the art shall certainly be able to achieve many other equivalent forms of device for male urinary incontinence, having the characteristics as set forth in the claims and hence all coming within the field of protection defined thereby.

In the following claims, the sole purpose of the references in brackets is to facilitate reading: they must not be considered as restrictive factors with regard to the field of protection claimed in the specific claims.

1. Device for male urinary incontinence comprising at least one pair of underpants (11) in which at least one through hole (18) is made, into which the urogenital apparatus (19) is introduced, and at least one absorbent aid (12) provided with at least one bag (21) and connected to the external surface (13) of the underpants (11) by means of support and attachment means (14, 17, 17′), said absorbent aid (12) being provided with at least one through hole (20) substantially aligned with the through hole (18) of the underpants (11) to allow the introduction of the urogenital apparatus (19) inside said bag (21) of the absorbent aid (12), characterized in that said underpants (1 1) comprise, on the external surface (13), at least one pocket (14) having an upper entry side for the insertion of a lower first portion (15) of the absorbent aid (12), said absorbent aid (12) comprising an upper second portion (16) outside the pocket (14) which is connected to the external surface (13) of the underpants (11) by means of removable attachment means (17, 17′).

2. Device as in claim 1, characterized in that at least one of either the through hole (18) or the through hole (20) is provided with a membrane (33) on which a plurality of cuts are defined passing through the center of said through hole (18, 20) and defining a coherent plurality of membrane sectors (33′) configured to allow the selective introduction of the urogenital apparatus (19) inside the bag (21) of the absorbent aid (12).

3. Device as in claim 1, characterized in that said through hole (18) of the underpants (11) comprises an elasticized edge (25).

4. Device as in claim 1, characterized in that said through hole (20) of the absorbent aid (12) comprises an elasticized edge (26).

5. Device as in claim 3, characterized in that at least one of either the elasticized edge (25; 26) is defined by an elastic stitching.

6. Device as in claim 1, characterized in that said absorbent aid (12) comprises, from inside to outside, at least one layer (29) of antiallergic and antibacterial material and at least one layer (30) of pressed cellulose with high carrying capacity.

7. Device as in claim 1, characterized in that said absorbent aid (12) comprises, from inside to outside, at least one layer (29) of antiallergic and antibacterial material, at least one layer (30) of pressed cellulose with high carrying capacity and at least one layer (31) of pressed cellulose made of super absorbent polymers.

8. Device as in claim 1, characterized in that said absorbent aid (12) comprises, from inside to outside, at least one layer (29) of antiallergic and antibacterial material, at least one layer (30) of pressed cellulose with high carrying capacity, at least one layer (31) of pressed cellulose made of super absorbent polymers and at least one layer (32) of hypoallergic-antibacterial pressed cellulose.

9. Device as in claim 1, characterized in that at least said absorbent aid (12) is externally coated by at least one waterproof external layer made of non-woven polypropylene fabric, water-repellent non-woven fabric.

10. Device as in claim 1, characterized in that said absorbent aid (12) comprises at least one pair of wings (27) on which the removable attachment means (17′) are positioned, said wings (27) being attached on one side and on the other of the second portion (16), since they are disposed outside the bulk of the second portion (16).

11. Device as in claim 1, characterized in that said pocket (14) is provided, on the entry side of the pocket (14), with an elasticized edge (28).

12. Underpants, comprising a through hole (18) into which the urogenital apparatus (19) is introduced and usable in a device for male urinary incontinence as in claim 1.

13. Absorbent aid (12), provided with at least one bag (21), at least one through hole (20) to allow the introduction of the urogenital apparatus (19) inside said bag (21) and usable in a device for male urinary incontinence as in claim 1.
